(19) United States
(12) Patent Application Publication
(10) Pub. No.: US 2015/0315536 A1
(43) Pub. Date: Nov. 5, 2015

(54) CELL CULTIVATING FLASK

(71) Applicants: David Alan KENNEY, Chelmsford, MA (US); Todd Michael TON, Eliot, ME (US); Joseph Christopher WALL, Southborough, MA (US); CORNING INCORPORATED, Corning, NY (US)

(72) Inventors: David Alan Kenney, Chelmsford, MA (US); Todd Michael Upton, Eliot, ME (US); Joseph Christopher Wall, Southborough, MA (US)

(21) Appl. No.: 14/647,558

(22) PCT Filed: Nov. 27, 2013
(86) PCT No.: PCT/US2013/072152

§ 371 Date: May 27, 2015

Related U.S. Application Data
(60) Provisional application No. 61/731,842, filed on Nov. 30, 2012.

(51) Int. Cl. C12M 1/24; C12M 1/12

(52) U.S. Cl. CPC C12M 23/08; C12M 25/06

(57) ABSTRACT

A flask for the culturing of cells is disclosed. The cell culture chamber is defined by top and bottom walls connected by side and end walls, one end wall shaped such that media can drain to a bottommost spot thereby allowing for the complete removal of media via a vertically oriented pipette inserted into the interior of the flask body through an open neck.

### CROSS-REFERENCE TO RELATED APPLICATIONS

This application claims the benefit of priority under 35 U.S.C. §119 of U.S. Provisional Application Ser. No. 61/731,842 filed on Nov. 30, 2012, the entire content of which is hereby incorporated by reference.

### TECHNICAL FIELD

The present disclosure relates generally to the cellular biological field and, in particular, to a cell cultivating flask.

### BACKGROUND

In vitro culturing of cells provides material necessary for research in pharmacology, physiology, and toxicology. The environmental conditions created for cultured cells should resemble as closely as possible the conditions experienced by the cells in vivo. One example of a suitable medium for culturing cells is a common laboratory flask such as demonstrated in U.S. Pat. No. 4,770,854 to Lyman. The cells attach to and grow on the bottom wall of the flask, immersed in a suitable sustaining media. The flask is kept in an incubator to maintain it at the proper temperature and atmosphere.

Desirably, flasks are stacked together in an incubator and a number of cultures are simultaneously grown. Small variations in the growth medium, temperature, and cell variability have a pronounced effect on the progress of the cultures. Consequently, repeated microscopic visual inspections are needed to monitor the growth of the cells. As such, cell culture flasks are typically constructed of optically clear material that will allow such visual inspection.

With the advent of cell-based high throughput applications, fully automated cell culture systems have been the subject of serious development work (see e.g. A Review of Cell Culture Automation, M. E. Kempner, R. A. Felder, JALA Volume 7, No. 2, April/May 2002, pp. 56-62.) These automated systems employ traditional cell culture vessels (i.e. common flasks, roller bottles, and cell culture dishes). These systems invariably require articulated arms to uncap flasks and manipulate them much like the manual operator. During such automated manipulation, it is often required to remove all media within a cell culture flask. Conventional flasks require angled manipulation of a pipette through the neck in order to fully remove all media. This limits the size of the pipette that may be employed.

There is a need for a cell culture flask having a rigid structure that is capable of use with any number of conventional pipette sizes while being suitable for use in the performance of high throughput assay applications that commonly employ robotic manipulation. There is also a need for a cell culture flask that can be fully emptied with a pipette from a single position.

### SUMMARY

According to an illustrative embodiment of the present disclosure, a flask for the efficient culturing of cells is disclosed. The illustrative flask includes a unitary body including a bottom wall defining a cell growth area and a top wall, connected by side walls and end walls. For the addition and removal of media, the flask is equipped with a wide neck defining an opening or aperture allowing access to the body of the flask itself. A sloped shoulder region is included which tapers toward the neck and enables pouring. The end wall opposite the neck is configured in such a way as to enable media pooling at a point directly opposed to the neck opening. This allows the removal of media from a single point opposite the neck opening when the flask is oriented with the neck opening facing upward. In addition, the flask of the present disclosure is shaped and configured to enable robotic access to the flask interior without requiring cumbersome robotic arm manipulation.

### BRIEF DESCRIPTION OF THE DRAWINGS

The disclosure is best understood from the following detailed description when read with the accompanying drawing figures. It is emphasized that the various features are not necessarily drawn to scale. In fact, the dimensions maybe arbitrarily increased or decreased for clarity of discussion.

FIG. 1 is a perspective view according to one embodiment.

FIG. 2 is a bottom view according to one embodiment.

FIG. 3 is a side view according to one embodiment.

FIG. 4 is a view of the use of a pipette with an illustrative embodiment.

### DETAILED DESCRIPTION

In the following detailed description, for purposes of explanation and not limitation, exemplary embodiments disclosing specific details are set forth in order to provide a thorough understanding of the present disclosure. However, it will be apparent to one having ordinary skill in the art that the present disclosure may be practiced in other embodiments that depart from the specific details disclosed herein. In other instances, detailed descriptions of well-known devices and methods may be omitted so as not to obscure the description.

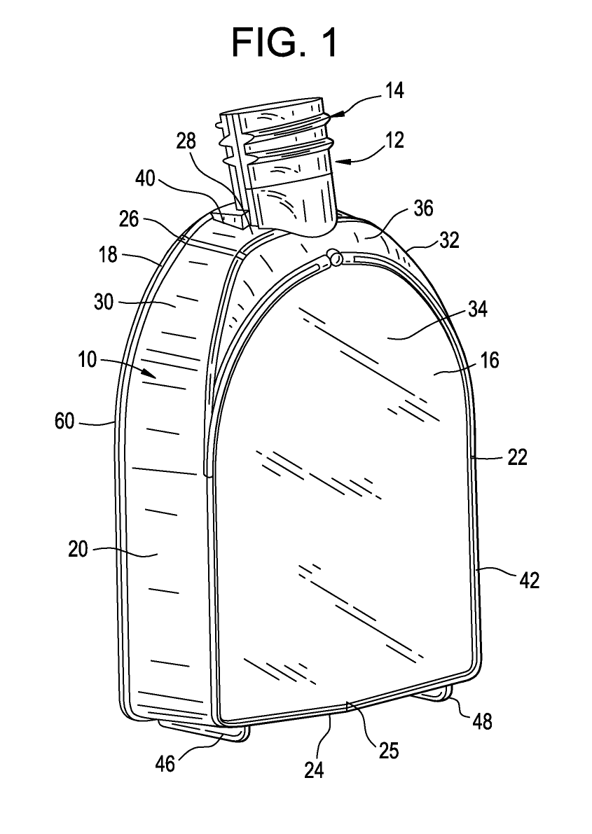

The laboratory flask shown in the drawing includes a body 10, a neck 12 and a threaded region 14 to receive a removable coverall screwcap. In this specification and when referring to top, bottom, sides, etc., the flask will be generally described in an orientation where the neck portion is in an approximate horizontal position and the bottom wall 16 facing the observer in FIG. 1 is in contact with a flat surface such as a laboratory bench. However, it is of course understood that the flask is not limited in any way to that particular position.

The body 10 has a bottom wall 16 and a top wall 18 that generally lie in parallel planes, and they are connected together by side walls 20 and 22, and first and second end walls 24 and 26. The neck 12 is integrally formed with the central section 28 of the second end wall 26. The second end wall 26 also includes diverging sloping portions 30 and 32 which extend from the ends of the central portion 28 to the side walls 20 and 22, respectively. The top wall 18 is essentially flat throughout its full extent. The bottom wall 16 throughout its major rectangular and arched portion 34 is flat and parallel to the top wall 18 while the remaining portion 36 of the bottom wall defines an inclined ramp from the neck 12 to horizontal, arched portion 34 of the bottom wall. The ramp 36 is disposed in an angle of approximately 15-30° with the horizontal while the margins of the ramp defined by the end wall portions 30 and 32 diverge from one another in a sloped arching fashion.

In FIGS. 1-3, the rectangular arching portion 34 of the bottom wall 16 is shown to carry a downwardly extending bead 42 about its periphery, which functions as one part of a stacking facility provided in the flask to enable identical flasks to be stacked compactly and positively with one another. The other part of the stacking facility is in the form of an upwardly extending flange 44 formed about the edge of the top wall 18. Because the plan dimensions of the top wall 18 slightly exceed the corresponding dimensions of the bottom wall 16, when one flask is stacked upon another, the bead 42 on the bottom wall just fits within the flange 44 on the top wall. There is an interruption in the flange 44 to prevent a vacuum when stacked or placed on a wet surface and in no way affects the stacking facility.

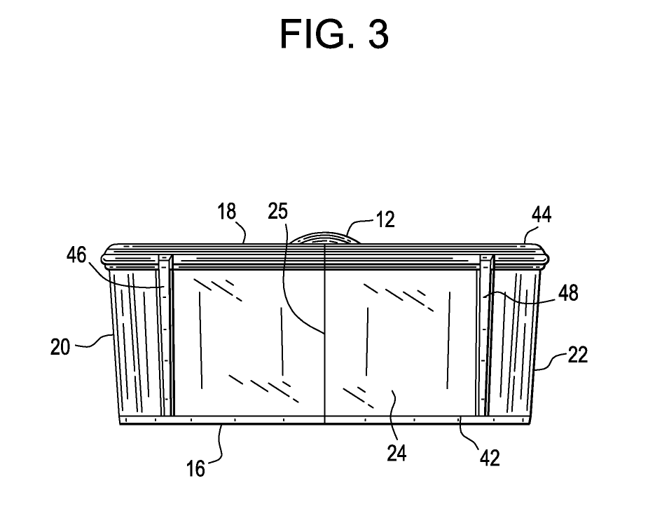

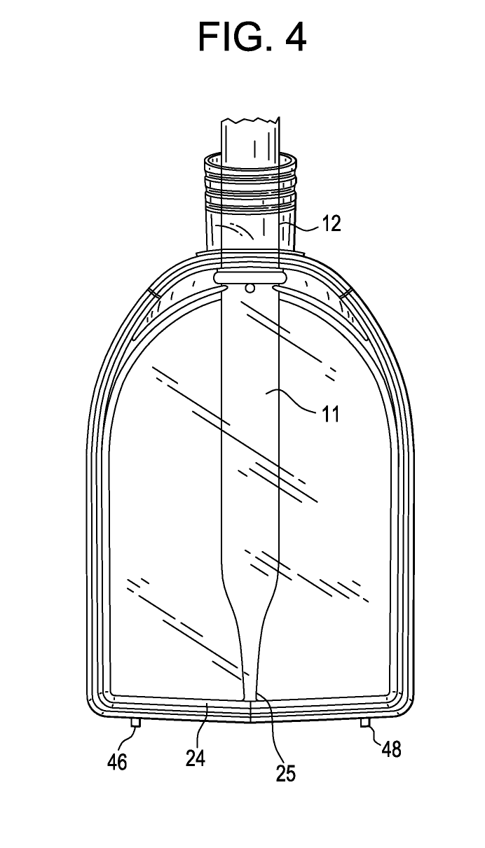

A sloping feature along the innermost portion of the first end wall 24 enables complete drainage and removal of media when the flask is arranged in position in which the neck 12 faces upward. In the embodiment disclosed and in the orientation illustrated in FIGS. 1, 2 and 4, the first end wall 24 is comprised of two opposed portions sloping downward at an angle of between 5-25° angle and together intersecting at an obtuse angle of less than 180 degrees, and in one embodiment between 150 and 175°. The focal point of the angle forms the bottommost point for liquid containment within the flask body in this orientation. As shown by FIG. 3, this focal point may take the form of a line 25. In such a position, media will pool along the line at the bottom most portion 25 of the sloped end wall, which is located in direct vertical alignment with the center of the screw cap neck. For example, in FIG. 4, a pipette 11 is shown entering the flask through the neck 12 and engaging the lowermost point 25 in the sloped inner portion of first end wall 24. In such a position, the neck opening can accommodate the largest possible size pipette and still be capable of draining the contents of the flask in its entirety. In other embodiments, there is both a slope in the x-axis as well as the y-axis of the first end wall thereby creating a single bottommost focal point in the end wall within which all remaining liquid within the flask body will pool.

The neck portion may be straight or canted. The diameter of the opening defined by the neck may be any size. In one embodiment, the opening ranges from approximately 25-35 mm. A generally accepted standard size for cell culture flasks is approximately 30 mm diameter fitting to a 33 mm cap.

Attached to the first end wall 24 are two feet 46, 48 that are shaped to accommodate any slope in the first end wall and will allow the flask to stand upright as demonstrated in the orientation displayed in FIGS. 1, 2 and 4.

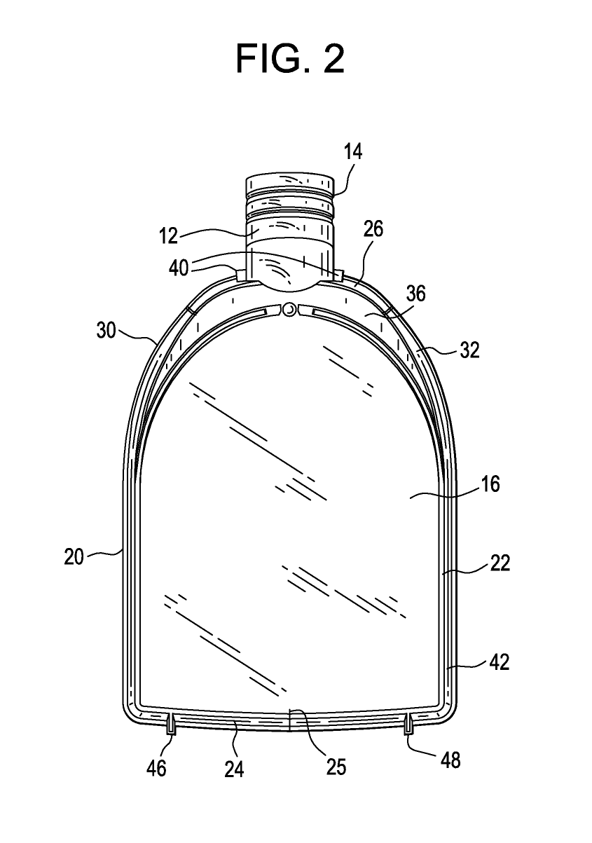

The flask is injection molded in two parts from a clear plastic material such as a polysterene. One part of the flask includes the bottom wall 16, side walls 20 and 22, end walls 24 and 26, ramp 36 and neck 12. The other part comprises the top wall 18, a short skirt 60 that fits over the top edges of the side and end walls 20, 22, 24 and 26, and a generally semi-circular collar 40 that surrounds the upper half of neck 12 as is shown in FIGS. 1 and 2. The collar 40 assists in positioning the top wall and skirt on the bottom part of the container when the two are cemented or otherwise secured together in sealed relationship. The collar 70 also serves to strengthen the connection between the neck 12 and the second end wall 26. It will be appreciated that a slight draft is provided in the side walls 20 and 22 and end walls 24 and 26 to facilitate removal of the lower part of the container from the mold during manufacture. This in turn results in the slightly larger surface for top wall 18 so as to provide a firm seat for the bottom wall of another flask when one is stacked upon another. The parts are held together and are adhesive bonded along the seam, ultrasonically welded, or scan welded. Preferably, scan welding equipment is utilized in a partially or fully automated assembly system. The two parts are properly aligned while a scan weld is made along the outer periphery of the joint. For flasks made with polystyrene, the thickness is preferably greater than 0.5 mm and more preferably greater than 1 mm. This thickness ensures that the flask bottom wall be perfectly flat, which in use provides a durable surface that will readily attach a uniform cell layer. Although not required, for optical clarity, it is advantageous to maintain a thickness of no greater than 2 mm.

Advantageously and in order to enhance cell attachment and growth, the surface of the bottom wall is treated to make it hydrophilic. Treatment may be accomplished by any number of methods known in the art which include plasma discharge, corona discharge, gas plasma discharge, ion bombardment, ionizing radiation, and high intensity UV light. Although cell attachment is typically targeted for only one surface (the inner potion of the bottom wall), other parts of the flask may be treated so as to enable cell growth on all surfaces of the flask interior.

The configuration of the flask provides several advantages. The fluid collection area enables a serological pipette to access the flask through the opening and since the low spot in the first end wall is located directly opposite the opening in the neck, no manipulation of the pipette is required to fully empty the flask of media. Pipette size will range with flask size, but generally, the following pipette sizes may be used with the relative associated flasks:
25 cm2 with pipettes 10 ml, 5 ml, 2ml, 1 ml
75 cm2 with pipettes 50 ml, 25 ml, 10 ml, 5 ml, 2 ml, 1 ml
150 cm2 with pipettes 50 ml, 25 ml, 10 ml, 5 ml, 2 ml, 1 ml
175 cm2 with pipettes 100 ml, 50 ml, 25 ml, 10 ml, 5 ml, 2 ml, 1 ml
225 cm2 with pipettes 100 ml 50 ml, 25 ml,10 ml, 5 ml, 2 ml, 1 ml

Such an arrangement also has benefits in automated cell growth procedures since conventional robots are more capable of a vertical pipette insertion and less prone or capable of angular pipette manipulation.

Finally, a cap (not shown) is provided, in one embodiment having a septum that is integral with the cap top. This will allow a cannula, tip or needle to access the contents of the flask without the need for unscrewing. The septum is leak proof, puncturable and capable of resealing once the needle, tip or cannula is removed from the flask, even after multiple punctures.

In use, the flask of the current disclosure is employed according to accepted cell growth methods. Cells are introduced to the flask though the threaded neck. Along with the cells, media is introduced such that the cells are immersed in the media. The flask is arranged such that the cell containing media covers the cell growth surface of the bottom wall. It is important not to completely fill the flask so as to allow for proper oxygenation of the media and cells. The flask is then placed within an incubator and maybe stacked together with similar flasks such that a number of cell cultures are simultaneously grown. The flask is situated such that the bottom wall assumes a horizontal position that will allow it to be completely covered by media. Cell growth is monitored from time to time by microscopic inspection through the bottom wall. During the cell growth process, it may become necessary to extract the exhausted media and insert fresh media. As previously described, media replacement may be achieved through insertion of a pipette, for example, through the opening in the neck. Once the cells are ready for harvesting, a chemical additive such as trypsin is added to the flask through the opening in the neck. The trypsin has the effect of releasing the cells from the flask walls. The cells are then harvested from the flask.

Being thus described, it would be obvious that the same may be varied in many ways by one of ordinary skill in the art having had the benefit of the present disclosure. Such variations are not regarded as a departure from the spirit and scope of the disclosure, and such modifications as would be obvious to one skilled in the art are intended to be included within the scope of the following claims and their legal equivalents.

1. A flask for the growth of cells comprising:
a flask body serving as a cell culture chamber defined by a bottom wall having a rigid surface and a top wall, the bottom wall and top wall connected by side walls and first and second walls,
a neck portion to the body open at one end and connected to the body at the other end providing access to the flask body;
wherein an interior portion of said first end wall is sloped such that when the flask is situated with the open end of the neck facing upwards, the lowest-most point of the interior portion of the first end wall is located in vertical alignment with the open end of the flask

2. The flask according to claim 1, wherein the neck opening has a diameter of between 25 and 35 mm.

3. The flask according to claim 1, further comprising a cap that fittingly engages the neck in order to seal the contents of the flask.

4. The flask according to claim 1 wherein said bottom portion further comprises a substantially flat region defined by a substantially rectangular portion and an arched portion.

5. The flask according to claim 4 wherein the bottom wall further comprises an included ramp connecting the neck to the substantially flat region.

6. The flask of according to claim 1 wherein said second end wall comprises diverging sloping portions which extend from a central portion of the side walls

7. The flask according to claim 1 further comprising feet affixed to an external portion of the first side wall.

8. The flask according to claim 1 wherein the interior portion of the first end wall comprises two sections meeting at a focal point and creating an angle, the angle being between 150 and 175 degrees.

9. The flask according to claim 1 further comprises a single bottommost point in the interior surface of the interior portion of the first end wall when the flask is oriented such that the opening in the neck is facing upward.

10. The flask according to claim 9 wherein the single bottommost point is located in direct vertical alignment with the center of the opening in said neck.

11. The flask according to claim 1 wherein the flask body holds up to 225 cm2 of liquid.

12. The flask according to claim 1 further comprising stand-offs either rising from an exterior surface of the top wall or descending from an exterior surface of the bottom wall.

13. The flask according to claim 5 wherein the ramp portion is disposed at an angle of between 15 and 30 degrees with the horizontal as defined by the bottom wall.

14. The flask according to claim 1 wherein an inner surface of the bottom wall is hydrophilic.

15. The flask according to claim 1 wherein the bottom wall has a thickness of between 0.5 mm to 2 mm.

16. The flask according to claim 3 further comprising a septum that is integral with the cap top.
